(19) United States
(12) Patent Application Publication
(10) Pub. No.: US 2012/0277587 A1
(43) Pub. Date: Nov. 1, 2012

(54) METHOD AND APPARATUS FOR REAL TIME MONITORING OF TISSUE LAYERS

(76) Inventors: Yossef Ori Adanny, Mitzpe Ilan (IL); Avner Rosenberg, Bet Shearim (IL); Edward Kantorovich, Rehovot (IL)

(21) Appl. No.: 13/393,212

(22) PCT Filed: Oct. 7, 2010
(86) PCT No.: PCT/IL10/00814

§ 371 Date: May 3, 2012

Related U.S. Application Data
(60) Provisional application No. 61/254,670, filed on Oct. 24, 2009.

(51) Int. Cl. A61N 7/00; A61B 8/00

(52) U.S. Cl. 600/439

(57) ABSTRACT

The disclosed method and apparatus employ ultrasound beams to monitor the tissue type composition of body tissue that is to be treated and the temperature at each body tissue type or layer in real time. Additionally, the disclosed method and apparatus also provides ultrasound-based thermo-control of an aesthetic body treatment session.

### CROSS-REFERENCE TO RELATED APPLICATIONS

This application is being filed under 35 U.S.C. 371 and claims the benefit of the filing date of U.S. provisional application for patent that was filed on Oct. 24, 2009 and assigned Ser. No. 61/254,670 by being a national stage filing of International Application Number PCT/IL2010/000814 filed on Oct. 7, 2010, each of which are incorporated herein by reference in their entirety. This application is related to U.S. patent application of Assignee that was filed on Jul. 15, 2009 and assigned Ser. No. 12/503,834, the disclosure of which is hereby incorporated by reference

### TECHNICAL FIELD

The method and apparatus relate to the field of aesthetic body shaping devices and more specifically to a method and apparatus for real time monitoring of tissue layers treated by aesthetic body shaping devices.

### BACKGROUND

Aesthetic body shaping devices are operative to effect treatment to the delicate body tissue layers by employing numerous methods of therapy. The methods apply various forms of energy to the tissue, one of which is thermotherapy, consisting of the application of heating energy into the tissue in a form of light, radiofrequency (RF), ultrasound, electrolipophoresis, iontophoresis and microwaves and any combination thereof.

Since all methods of thermotherapy increase the tissue temperature to about 40-60 degrees Celsius, monitoring of the tissue temperature and the type of tissue layers being treated is imperative. Methods used in the art characteristically monitor treated body tissue temperature employing sensors such as thermocouples or thermistors incorporated in electrodes or transducers through which the energy is applied to the skin. Other methods employ ultrasound monitors that determine temperature changes based on ultrasound echo reflection and deflection.

Many aesthetic body shaping methods also employ vacuum chambers. Suction in the vacuum chamber draws tissue to be treated into the chamber and treating energy is applied to the tissue. Commonly, aesthetic body shaping device applicators are coupled to a tissue segment to be treated without careful monitoring of the composition of the tissue layers constituting the segment. This may result in drawing into the vacuum chamber tissue layers not intended to be treated, such as muscle, and applying heating energy resulting in irreversible damage thereto.

Commonly, ultrasound echo imagery may also be employed during aesthetic body shaping sessions to follow the course of the treatment session by employing quantitative monitoring of primarily only the fat tissue layer being treated.

Currently, employed monitoring methods, as mentioned hereinabove, do not monitor temperature in discrete tissue layers.

### BRIEF SUMMARY

The disclosed method and apparatus employ ultrasound beams to monitor the tissue type composition of body tissue to be treated and temperature at each body tissue type or layer in real time. Additionally, the disclosed method and apparatus also provides ultrasound-based thermo-control of an aesthetic body treatment session.

In accordance with an exemplary embodiment of the disclosed method and apparatus an applicator includes a housing, an ultrasound beam first transducer, operative to emit ultrasound beams into a segment of tissue and a second transducer operative to receive the emitted beams. The first transducer and second transducer each consist of one or more piezoelectric elements. Additionally or alternatively, each of the first and second transducers may emit and/or receive ultrasound beams.

In accordance with yet another exemplary embodiment of the disclosed method and apparatus the housing may also include a vacuum chamber that employs vacuum to draw the segment of tissue into the chamber. In accordance with still another exemplary embodiment of the disclosed method and apparatus the chamber walls may also be operative to shift a propagation pathway of emitted ultrasound beams from a first propagation pathway to a second propagation pathway parallel thereto. This allows monitoring tissue composition and temperature in remote tissue areas previously not monitored due to physical constraints such as the at the apex of a tissue protrusion inside the vacuum chamber.

In accordance with another exemplary embodiment of the disclosed method and apparatus the transducer elements may be arranged in one or more two-dimensional or three-dimensional spatial configurations. The first transducer may be operative to emit ultrasound beams in pulse form through a tissue protrusion to be treated. A controller may be employed to obtain information from ultrasound beams received from the second transducer, and communicated therefrom. Such information may include changes in propagation speed, amplitude and attenuation. The controller may analyze the information to determine tissue composition (E.g., skin and fat, fat and muscle, etc) and layer type (E.g., skin, fat, muscle, etc.) and temperature at each tissue type or layer prior to and during a treatment session.

In accordance with yet another exemplary embodiment of the disclosed method and apparatus the controller may also be operative to obtain from received ultrasound beam signals information including changes in beam propagation speed through a discrete tissue layer and analyze the information to determine tissue layer type (E.g., skin, muscle or fat) and changes in tissue layers composition (E.g., penetration of muscle layer into fat tissue layer being treated, etc.) in real time.

In accordance with still another exemplary embodiment of the disclosed method and apparatus the controller may communicate the changes in treatment parameters, to a power generator. The generator may cease or initiate excitation of the first transducer, or, alternatively, may change the level of excitation, in accordance with input received from the apparatus controller.

In accordance with another exemplary embodiment of the disclosed method and apparatus the applicator may also employ one or more sources of heating energy in a form of at least one of a group consisting of light, radiofrequency (RF), ultrasound, electrolipophoresis, iontophoresis and microwaves.

### BRIEF DESCRIPTION OF THE DRAWINGS

The disclosed method and apparatus will be understood and appreciated from the following detailed description, taken in conjunction with the drawings in which:

FIG. 1A and FIG. 1B are simplified cross-sectional views, at right angles to each other, illustrating an exemplary embodiment of the disclosed method and apparatus employed in an aesthetic body treatment applicator vacuum chamber to monitor composition and/or temperature of a tissue treatment area.

FIG. 2 is a simplified cross-sectional view illustrating another exemplary embodiment of the disclosed method and apparatus employed in a vacuum chamber of an aesthetic body treatment applicator to monitor composition and/or temperature of a remote tissue treatment area.

FIG. 3A, FIG. 3B and FIG. 3C are simplified illustrations of a configuration of the piezoelectric elements in yet another exemplary embodiment of the disclosed method and apparatus employed in a vacuum chamber of an aesthetic body treatment applicator to monitor composition and/or temperature of a tissue treatment area.

FIG. 4A and FIG. 4B are simplified illustrations of a configuration of the piezoelectric elements in a first and second transducers and block diagrams of the electronic system for the control thereof in accordance with still another exemplary embodiment of the disclosed method and apparatus.

FIG. 5 is a simplified block diagram of a configuration of the electronic system of another exemplary embodiment of the disclosed method and apparatus employed in a vacuum chamber of an aesthetic body treatment applicator, such as that in FIGS. 1A and 1B and/or 3A and 3B, to monitor composition and/or temperature of a tissue treatment area.

FIG. 6 is a graph depicting a signal of a received ultrasound beam pulse in accordance with still another exemplary embodiment of the disclosed method and apparatus.

FIG. 7A, FIG. 7B, FIG. 7C and FIG. 7D are simplified views illustrating ultrasound wave propagation in accordance with an exemplary embodiment of the disclosed method and apparatus.

### GLOSSARY

The terms “transducer” and “transceiver” as used in the present disclosure mean energy conversion devices, such as piezoelectric elements, that emit and/or receive ultrasound beams and may be used interchangeably, their functionality (such as emitting or receiving ultrasound beams) defined by their predetermined location in the apparatus and electric connection to a controller as will be described in detail below.

The term “body tissue” in the present disclosure means any superficial body tissue layer, primarily one or more of the following body tissue layers: Skin, fat and muscle.

The term “cylinder” as used in the present disclosure means a three-dimensional shape with straight parallel sides and a cross section selected from a group of geometrical shapes such as a circle, a square, a triangle, etc.

### DETAILED DESCRIPTION OF THE EXEMPLARY EMBODIMENTS

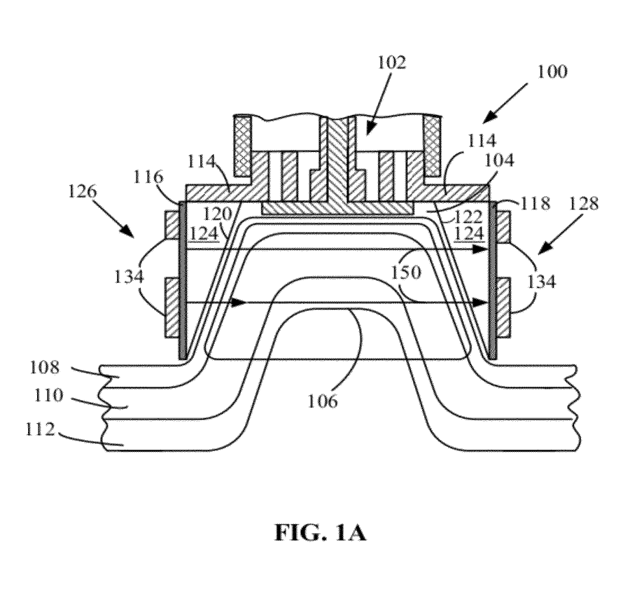

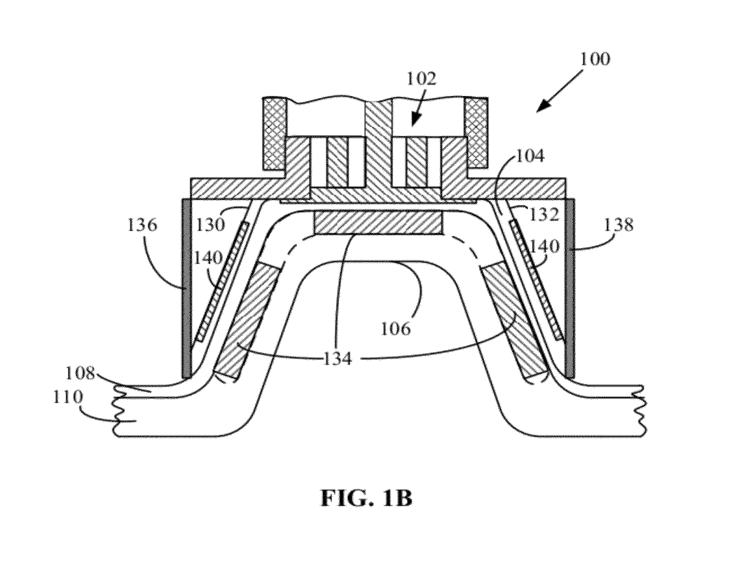

Reference is now made to FIG. 1A and FIG. 1B which are simplified cross-sectional views, at right angles to each other, illustrating an exemplary embodiment of the disclosed method and apparatus employed in an aesthetic body treatment applicator vacuum chamber to monitor composition and/or temperature of a tissue treatment area.

Applicator 100 includes a housing 102 including one or more vacuum chambers 104, which, for example, may be of the type disclosed in assignee's U.S. patent application of Assignee that was filed on Jul. 15, 2009 and assigned Ser. No. 12/503,834, the disclosure of which is hereby incorporated by reference. A tissue protrusion 106 to be treated, including body tissue layers: skin 108, fat 110 and muscle 112, is located within vacuum chamber 104.

In an exemplary embodiment of the disclosed method and apparatus, housing 102 is a cylinder having a first end sealed by a closed portion 114 and a second open end and defined by one or more walls 116, 118, 136 and 138 (FIG. 1B) also enveloping vacuum chamber 104.

Chamber 104 is defined by closed portion 114 of housing 102 and one or more walls 120, 122, 130 and 132 and the surface of skin tissue layer 108.

Each pair of walls 116 and 120, and 122 and 118 defines between them a cavity 124. Cavities 124 may be filled with any ultrasound matching material known in the art such as water, gel, oil or polyurethane.

Walls 116, 118, 136 and 138 as well as walls 120, 122, 130 and 132 may made of a polymer resin such as polyetherimide known as Ultem® 1000, manufactured by General Electric Advanced Materials, U.S.A. (see the URL at).

A first ultrasound transducer 126 and a second ultrasound transducer 128, each consisting of one or more piezoelectric elements 134, are positioned on the outside surface of walls 116 and 118 respectively. First ultrasound transducer 126 is operative to emit ultrasound beams into tissue protrusion 106 before, during or following a treatment session. Second transducer 128 is operative to receive ultrasound beams emitted by transducer 126, propagated in a substantially direct pathway through tissue protrusion 106 and emitted thereby (The Figure is schematic and does not show ultrasound beam refraction at the different boundaries.). Ultrasound transducer 128 is positioned facing transducer 126 at a predetermined distance from and substantially parallel to, so that transducers 126 and 128 sandwich protrusion 106 tissue layers 108, 110 and 112.

First transducer 126 emits ultrasound beams that propagate in a generally direct manner, along a pathway indicated by arrows 150, through wall 116, cavity 124, vacuum chamber wall 120, through tissue protrusion 106, continue through vacuum chamber wall 122, cavity 124, and wall 118 and are received by second transducer 128. Alternatively, in accordance with another exemplary embodiment of the method and apparatus, wall pairs 116 and 120, and 122 and 118 may be operative to shift the pathway of the ultrasound beams from a first propagation pathway to a second propagation pathway parallel thereto as will be described in detail below.

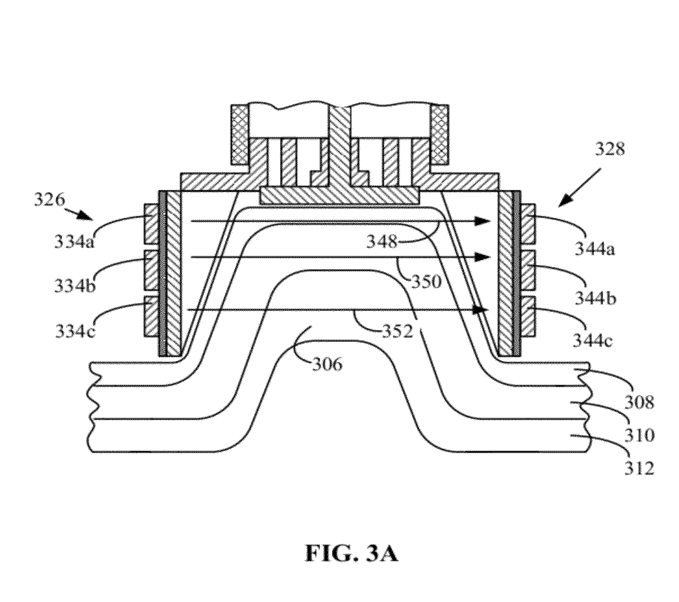

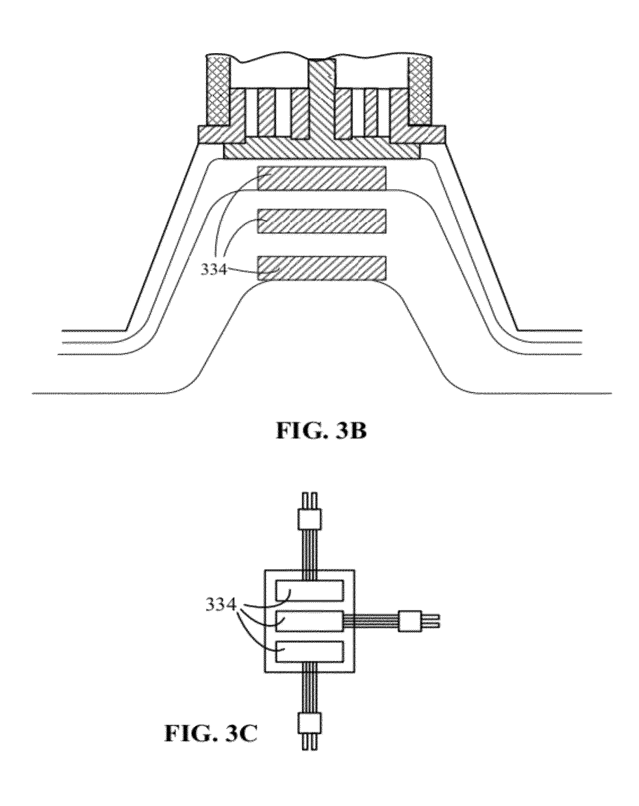

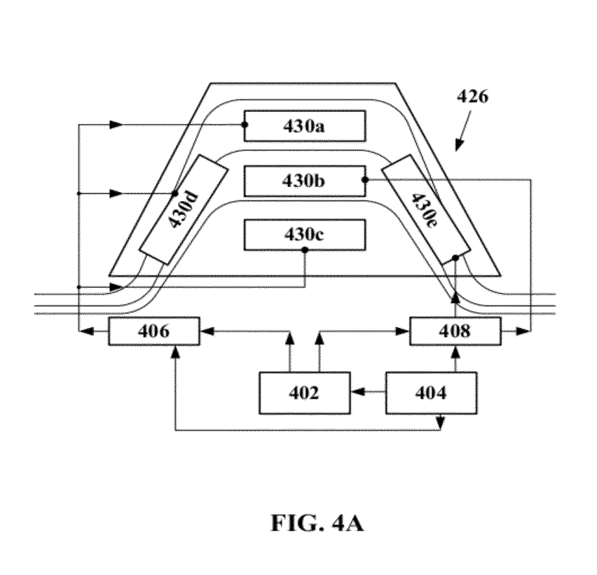

Piezoelectric elements 134 of transducers 126 and 128 may be constructed from one or more piezoelectric materials selected from a group consisting of ceramics, polymers and composites and may be positioned in one or more predetermined configurations selected from a group consisting of two-dimensional and three-dimensional spatial configurations. For example, In FIGS. 1A and 1B piezoelectric elements 134 are positioned on a single plane forming a two-dimensional arced configuration. In FIGS. 3A and 3B piezoelectric elements 334 are also positioned on a single plane forming a two-dimensional parallel configuration.

The amount of information that may be extracted from a signal depends on the pulse shape. The shorter the rise time (few nanosecond) the larger amount of information it may provide. The source of acoustic waves and its size should be selected to enable generating such pulses. In accordance with an exemplary embodiment of the disclosed method and apparatus elements 134 are made of polymeric materials possessing piezo electric properties and in particular Polyvinylidene Fluoride (PVDF). Another embodiment may use piezocomposite materials, which are compositions of ceramics and polymers. The selection of PVDF allows generation of a wide spectrum of wavelengths and an ultrasound pulse with a short pulse signal rise time. This allows receiving the most amount of information regarding the behavior of the beam propagation inside the tissue layer (for example, speed of sound, amplitude, frequency and/or attenuation). The received information may be further analyzed to identify the type of tissue the beam has propagated through and temperature thereof. The pulse signal rise time may be less than 200 ns, typically less than 100 ns and more typically less than 50 ns. The received centerline (acoustic axis) frequency spectrum may be between 500 KHz and 10 MHz, typically between 1.5 MHz and 4 MHz and more typically between 2.5 MHz and 3.5 MHz.

The thickness of a PVDF element, which is commercially available in thicknesses of 8 micron to 220 micron, affects the bandwidth of the ultrasound beam. Typically, the thickness of the piezoelectric element (D) is configured to be smaller than half the wavelength (λ) at the maximal frequency (f) so that

D<½λ at(fₘₐₓ).

Additionally, a lower thickness allows for larger capacitance of the piezo element supporting generation of acoustic energy at a lower voltage value. For example, PVDF thickness of 8 microns may provide a bandwidth of up to 25 MHz. In accordance with an exemplary embodiment of the disclosed method and apparatus the typical bandwidth may be about 15 MHz, and more typically 10 MHz and more typically 3 MHz. The PVDF element thickness to provide such bandwidth values is typically less than 500 microns and more typically less than 250 microns, less than 100 or more typically less than 50 microns.

Due to the physical-electrical nature of piezoelectric materials, it will be appreciated that transducers 126 and 128 may each also function as a transceiver, emitting an ultrasound beam when excited by electrical voltage received from a generator or converting a received ultrasound beam into an electrical signal communicated to a controller. The functionality of transducers 126 and 128 may be dependent on the electrical circuitry configuration of apparatus 100 or determined by a controller (not shown) controlling the directionality of the transmitted ultrasound beams from transducer 126 to transducer 128 or vice versa. Additionally and alternatively, transducers 126 and 128 may be operative to function as transceivers by each consisting of at least one element 134 operative to emit ultrasound beams and at least one element 134 operative to receive ultrasound beams.

According to another exemplary embodiment of the disclosed method and apparatus the controller is also operative to obtain information from transducer 128 regarding changes in speed of sound, amplitude, frequency and attenuation and analyze the information to determine tissue composition (e.g., skin and fat, fat and muscle, etc), layer type (e.g., skin, fat, muscle, etc.) and temperature at each tissue layer prior to and during a treatment session. The controller then may compare the tissue layer type or changes of temperature therein to a predetermined treatment protocol and determine the compatibility of the identified tissue layer type with the pending treatment to be applied to the body tissue and/or criticality of changes in the body tissue layers temperature, resulting in taking one or more actions based on the changes and criticality. Such actions may be, for example, one or more of the following: Record information relating to the changes and criticality in a database, display the information on a display, communicate the changes and criticality to a remote user, print the information on a printout, alert a user as to the changes based on their criticality and change the course of treatment based on the criticality.

The controller may also be operative to control each element 134 in transducers 126 and 128 individually and determine the sequence of ultrasound beam pulse delivery.

In the exemplary embodiment of the disclosed method and apparatus illustrated in FIG. 1B, walls 130 and 132 of vacuum chamber 104 also include heating energy delivery surfaces 140 positioned on the inner surfaces thereof. Heating energy delivery surfaces 140 are operative to apply heating energy in one or more forms selected from a group consisting of light, radiofrequency (RF), ultrasound, electrolipophoresis, iontophoresis and microwaves. Transducers 126 and 128 may also be positioned in a plurality of predetermined configurations in relation to heating surfaces 140, such as, for example, substantially perpendicular to energy delivery surfaces 140 or on the same plane and adjacent to energy delivery surfaces 140.

Another exemplary embodiment of the disclosed apparatus may also employ a method of applying RF energy to skin tissue layer 108 while concurrently externally cooling the surface thereof by, for example, employing heat conductive liquid media, for example, as described in assignee's U.S. Patent Application Publication number 2006/0036300.

In accordance with another exemplary embodiment of the disclosed method and apparatus the planes along which the elements of transducers 126 and 128 are arranged are substantially in parallel to each other and generally perpendicular to the surface of skin tissue layer 108 in its relaxed state (e.g., outside chamber 104), whereas the faces of walls 120, 122, 130 and 132 are slanted to provide increased comfort to a subject having aesthetic treatment. The angle of the slant may be dependent on the subject's skin characteristics. Firm and tight skin may require a greater slant and/or shallower chamber depth than looser more resilient skin that may conform more easily to lesser slanted chamber walls. Cavity 124 formed by the difference between the walls' spatial orientations, gaps the distance between the surfaces of transducers 126 and 128 and the surface of chamber walls 120 and 122 and tissue protrusion 106 drawn against the inside surfaces thereof. The presence of cavity 124 necessitates providing an index-matching medium therein, between transducers 126 and 128 and walls 120 and 122 respectively so that to minimize acoustic losses and maintain the desired direction and speed of acoustic wave propagation and improve transducer efficiency as will be explained in greater detail herein.

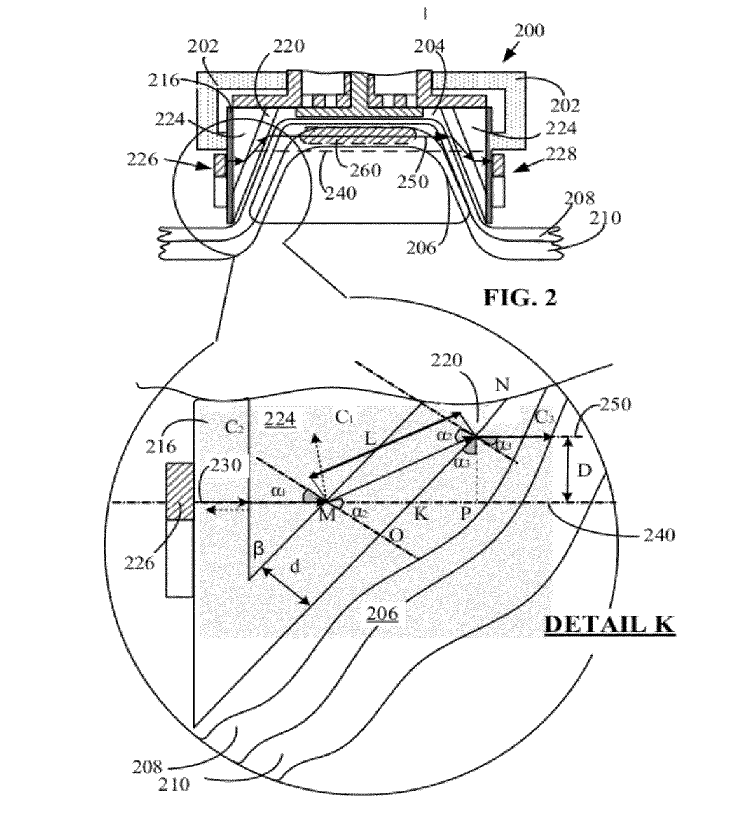

Reference is now made to FIG. 2, which is a simplified cross-sectional view of another exemplary embodiment of the disclosed method and apparatus employed in a vacuum chamber 204 of an aesthetic body treatment applicator 200 to monitor a remote tissue treatment area such as tissue area 260 located at the tip of a tissue protrusion 206.

FIG. 2 illustrates applicator 200 including a housing 202, a first transducer 226 and a second transducer 228. A treatment area 260 is located at the crest of protrusion 206. Alternatively, the treatment area may be located, for example, approximately 0.5 to 1 cm deep to the surface of skin tissue 208 (not shown) when at a relaxed (resting) state.

The most accurate information received is obtained from an ultrasound beam centerline as will be described in detail below. In such a configuration, the centerline of an emitted ultrasound beam may be refracted to propagate through the desired tissue area (e.g., at the crest of protrusion 206 or deep to skin layer 208).

Refraction shifts the pathway of the ultrasound beams emitted by transducer 226, from a first propagation pathway 240 to a second propagation pathway 250 parallel thereto, and re-shifting the pathway of the ultrasound beams from second propagation pathway 250 back to first propagation pathway 240 to be received by transducer 228, allows accurate monitoring tissue layer 210 type and/or the temperature of treatment area 260 at the tip of protrusion 206 and allowing greater flexibility in selecting the skin tissue layers and/or segments to be monitored. This also ensures ultrasound beam propagation substantially directly from transducer 226 to transducer 228 as will be explained in greater detail below.

Detail K is an enlargement of a portion of FIG. 2 and illustrates shifting of an emitted ultrasound beam 230 from a first propagation pathway 240 to a second propagation pathway 250 parallel thereto. In Detail K, (C1) represents the speed of sound in cavity 224, (C2) represents the speed of sound in walls 216 and 220 assuming that walls 216 and 220 are made of the same material (e.g., Ultem® 1000), and (C3) represents the speed of sound inside tissue protrusion 206. Alternatively, walls 216 and 220 may also be made of other materials to allow sound propagation at a plurality of predetermined velocities. Cavity 224 may be filled with any ultrasound sound index-matching material as known in the art and described in detail below.

The acoustic properties of index-matching material in cavity 224 such as acoustic impedance, dictate the behavior of the beam travelling therethrough affecting parameters such as speed of sound and refraction angle. Hence, the matching material properties, such as impedance, need to be similar to those of the tissue being monitored so that to minimize attenuation (i.e., loss or distortion of information) and refraction of ultrasound waves. Such refraction may occur when crossing, for example, the borders between, for example, housing wall 230 and cavity 224 and/or cavity 224 and chamber wall 220 and/or chamber wall 220 and the surface of tissue protrusion 206. For example, the impedance of human tissue is approximately 1.5 MRayl (Rayleigh). Materials such as castor oil, and more so water, have an acoustic impedance of approximately 1.4-1.5 MRayl. This allows the ultrasound beams to propagate in parallel to the tissue layers with minimal acoustic attenuation, reflection and refraction. Such materials may also include wedge type inserts such as plastics or polyurethane. Polymer materials such as polyurethane, which also have acoustic impedance close to that of the human body tend to create high attenuation at the upper part of the spectrum. A wedge made of thin walls of plastic and filled with water has the lowest attenuation over the spectrum of interest as described above. The temperature of the matching wedge and its filling may also be monitored and controlled employing a thermocouple and the temperature value incorporated into the wave propagation parameter analysis. Additionally and alternatively, the temperature of the matching material may be controlled by heating or cooling.

In another exemplary embodiment of the disclosed method and apparatus, the value of (D), which is the shifting distance between the original ultrasound beams propagation pathway 240 and desired propagation pathway 250 may be determined using the following expressions:

### Assuming C1=C3 (α₁=α₃):

$$\begin{matrix}L = \frac{d}{cos {α}_{2}} & ( 1 ) \\ \frac{{C}_{1}}{{C}_{2}} = \frac{sin {α}_{1}}{sin {α}_{2}} & ( 2 )\end{matrix}$$

From expressions (1) and (2):

$$L = \frac{d}{\sqrt{1 - \frac{{C}_{2}^{2}}{{C}_{1}^{2}} {sin}^{2} {α}_{1}}}$$

Since OK=d*tan α₁ and ON=\sqrt{L²−d²} then:

KN=ON−OK=\sqrt{L²−d²}−d*tan α₁

Extrapolating (D) from the above:

D=NP=KN*cos α₃=KN*cos α₁

or

$$D = ( \sqrt{\frac{{d}^{2}}{1 - \frac{{C}_{2}^{2}}{{C}_{1}^{2}} {sin}^{2} {α}_{1}} - {d}^{2}} - d * tan {α}_{1} ) * \sqrt{1 - {sin}^{2} {α}_{1}}$$

It will be appreciated from the above expressions that the distance (D) is dependent on several factors such as, among others, the composition of the vacuum chamber wall 220 and the refractive indexes of the materials composing the walls, the angle (α₂) which is also a derivative of the angle (β) between housing wall 216 and chamber wall 220 and the thickness of wall 220, the matching material in cavity 224 and temperature thereof. These factors may be predetermined and some may be adjusted to the desired area to be monitored in accordance with the type of treatment session to be applied.

Reference is now made to FIG. 3A and FIG. 3B, which are simplified cross-sectional views, at right angles to each other, of the configuration of the piezoelectric elements in yet another exemplary embodiment of the disclosed method and apparatus employed in a vacuum chamber of an aesthetic body treatment applicator for the identification of the tissue layers being treated and/or temperature thereof.

In the disclosed exemplary embodiment, a first transducer 326 and a second transducer 328 piezoelectric elements 334 and 344, respectively, are arranged in an array of three parallel elements positioned on one plane in a two-dimensional configuration. In this configuration, the elements are not only parallel to each other but also each of the corresponding pairs 334a-344a, 334b-344b and 334c-344c, sandwiches a segment of tissue, a major portion of which is occupied by one discrete tissue layer. For example, in FIG. 3A, the pair of elements 334a and 344a sandwiches a discrete segment of tissue consisting solely of tissue layer 308. The pair of elements 334b and 344b sandwiches a segment of tissue consisting mostly of tissue layer 310 and a small portion of layer 308. The pair of elements 334c and 344c sandwiches a segment of tissue consisting mostly of tissue layer 312 and small portions of tissue layers 308 and 310.

Each of elements 334 and 344 is located at a predetermined depth and configured as explained hereinabove to have the appropriate dimensions in accordance with tissue type, wedge matching material, etc. This allows information from each beam emitted by transducer 326 element 334 to be received individually by its corresponding transducer 328 element 344. This provides accurate treatment tissue type identification and heating temperature measurement at generally each of layers 308, 310 and 312 as indicated by arrows 348, 350 and 352 respectively.

In FIG. 3C, a simplified illustration of a three-element transceiver and the connectors thereof in accordance with another exemplary embodiment of the disclosed method and apparatus. Each of the three piezoelectric elements 334 may be operative to emit or receive ultrasound beams dependent on the electrical circuitry configuration of the apparatus or as determined by a controller (not shown).

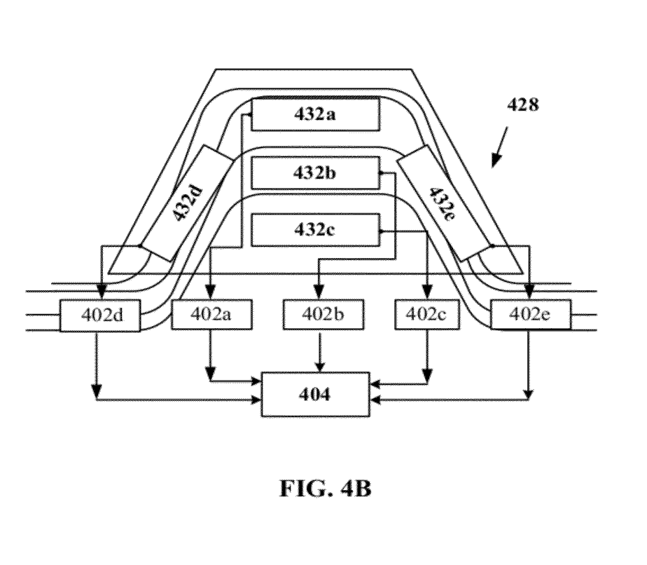

Reference is now made to FIGS. 4A and 4B, which are simplified illustrations of an example of a configuration of a first transducer 426 and a second transducer 428 piezoelectric elements 430a-430e and block diagrams of the electronic system for the control thereof in accordance with still another exemplary embodiment of the disclosed method and apparatus.

FIG. 4A illustrates transducer 426, the elements 430a-430e of which are arranged in a configuration combining an arced configuration such as that in FIG. 1B and a parallel configuration such as that in FIG. 3B.

A generator 402 generates power in accordance with input received from a controller 404. According to an exemplary embodiment of the disclosed method and apparatus, controller 404 may also synchronize the excitation of piezoelectric elements 430a, 430b, 430c, 430d and 430e through pulsers 406 and 408, or, alternatively through switches (not seen) in accordance with information obtained from received ultrasound beams regarding changes in propagation speed, amplitude and attenuation and analysis thereof and with a provided treatment protocol as described above.

In another exemplary embodiment of the disclosed method and apparatus, the element configuration described hereinabove may be used to determine several different parameters concurrently such as tissue layer temperature change and tissue layer type. In this case, for example, elements 430a, 430b and 430c may be employed to determine tissue layer type as described in FIG. 3 hereinabove, whereas elements 430d and 430e may be employed to measure treated tissue layer temperature.

FIG. 4B is a simplified illustration of an example of a configuration of second transducer 428 elements 432a-e and a block diagram of the electronic system for the control thereof in accordance with yet another exemplary embodiment of the disclosed method and apparatus. FIG. 4B illustrates elements 432a, 432b, 432c, 432d and 432e arranged in a configuration mirroring the configuration of elements 430a-e in transducer 426 (FIG. 4A). Each of elements 432a-e receives ultrasound beams emitted from their corresponding first transducer elements 430a-e which are then converted to a signal amplified by corresponding preamplifiers 402a-e and communicated individually to a controller 404 for analysis as described hereinabove.

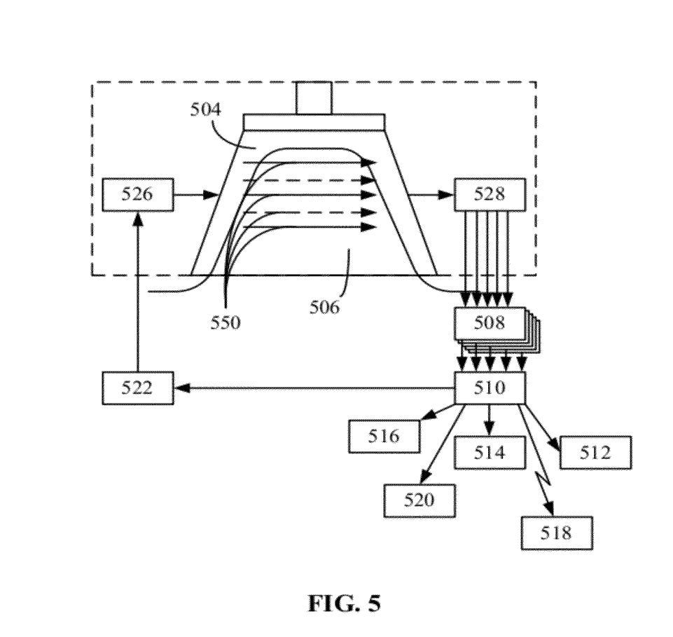

Reference is now made to FIG. 5, which is a simplified block diagram of a configuration of the electronic system of still another exemplary embodiment of the disclosed method and apparatus employed in a vacuum chamber 504 of an aesthetic body treatment applicator, such as that in FIGS. 3A and 3B, for the identification of the tissue layers being treated and/or temperature thereof.

Piezoelectric elements (not shown) of a first transducer 526, arranged in one or more of the configurations described hereinabove, emit ultrasound beams through a tissue protrusion 506 treated in vacuum chamber 504, as indicated by arrows 550. The emitted ultrasound beams received by a second transducer 528 are converted to signals amplified by preamplifiers 508.

The amplified electric pulses are communicated to a controller 510, operative to obtain from the received ultrasound beam signals information regarding changes in speed of sound, amplitude, frequency and attenuation, analyze the information to determine at least one tissue characteristic such as tissue layer type and/or treatment effect such as tissue layer temperature and take appropriate action.

Such actions may include one or more of the following: record information relating to the changes and criticality in a database 512, display the information on a display 514 such as a computer monitor or apparatus display, print the information on a printout 516, communicate the changes and criticality thereof to a remote user 518 or alert a user employing an alert 520 such as sounding an alarm, activating a warning light or any other type of alert, and change the course of treatment based on the criticality, as described hereinabove by, for example, increasing or decreasing the level of treatment heating energy application, changing the duration of treatment heating energy application or stopping the treatment session altogether. Controller 510 communicates the desired changes in treatment parameters, resulting from the determined criticality categorization to an electric power generator 522, which, accordingly, initiates, changes the level of or ceases, excitation of the elements of first transducer 526.

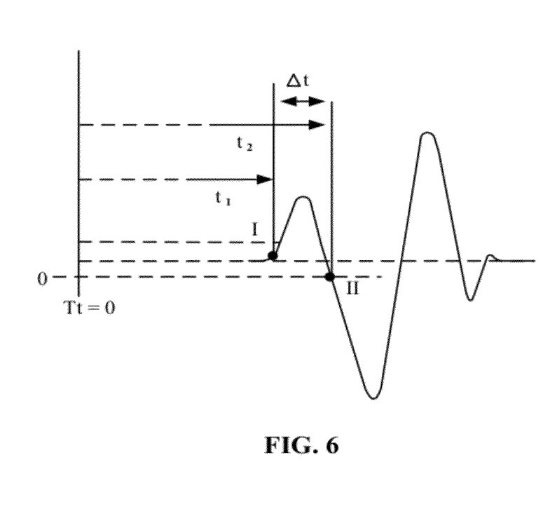

Reference is now made to FIG. 6, which is a graph depicting a sinusoidal signal of a received ultrasound beam pulse in accordance with yet another exemplary embodiment of the disclosed method and apparatus.

The speed of sound wave propagation through various body tissues is well documented and may also be achieved empirically. It is also well documented that propagation speed of sound beams through tissue is temperature-dependent and is altered by any increase or decrease in tissue temperature. The approximated values of speed of sound in tissue at normal body temperature are as follows:

Skin: Velocity (V)˜1700-1800 Meters per Second (m/s)

Fat: V˜1460 m/s; and

Muscle: V˜1580 m/s

FIG. 6 depicts a signal of a beam pulse, emitted at a known time (Tτ=0) and received at point (I) at signal receiving time (τ₁). Beam signal propagation time can thus be easily calculated by using the following expression:

V=L/τ₁

However, determination of the exact location of point (I) is inaccurate and a calibrated error coefficient must be factored into the calculation. This method is commonly practiced by person skilled in the art as the sole method for determining ultrasound beam propagation speed.

In accordance with an exemplary embodiment of the disclosed method and apparatus the accuracy of ultrasound beam propagation speed calculation is increased by recording the signal receiving time (τ₂) at the first signal zero-crossing point, indicated in the graph of FIG. 6 as point (II). Measurement of the distance between points (II) and (I) and factoring in the aforementioned calibrated error coefficient reduces the speed measurement error from relying on point (I) alone and provides a highly accurate calculation of the ultrasound pulse propagation speed. At a constant tissue temperature, consecutive transmitted pulses will retain their properties, such as length and amplitude, since the first transducer-second transducer distance is known and remains unchanged. Also, at such a short time interval, between signal transmission and reception, ultrasound beam dispersion is infinitesimally small. A change in tissue temperature changes the propagation speed of ultrasound beams thus increasing or decreasing the point (II)-Point (I) gap, increasing or decreasing the difference Δτ=(τ₂)−(τ₁). This difference can be easily extrapolated, for example, by an empirically derived reference table, to determine the tissue temperature change. For example, increase in tissue temperature allows a lower ultrasound beam propagation thus increasing the point (II)-Point (I) gap.

Information such as tissue layer type may be also achieved, not only from changes in beam propagation speed but also from changes in signal amplitude and the attenuation of the beam signal. The degree of change and criticality thereof may be extrapolated from comparing the information to one or more data references such as lookup tables (LUT) or data achieved empirically.

Analyzing the first signal received allows for time separation between received signals. This allows employing the same transducer to monitor composition and/or temperature of discrete tissue layers without interference between adjacent beams as will be described in detail hereinbelow. Typically pulse repetition is less than 10 kHz.

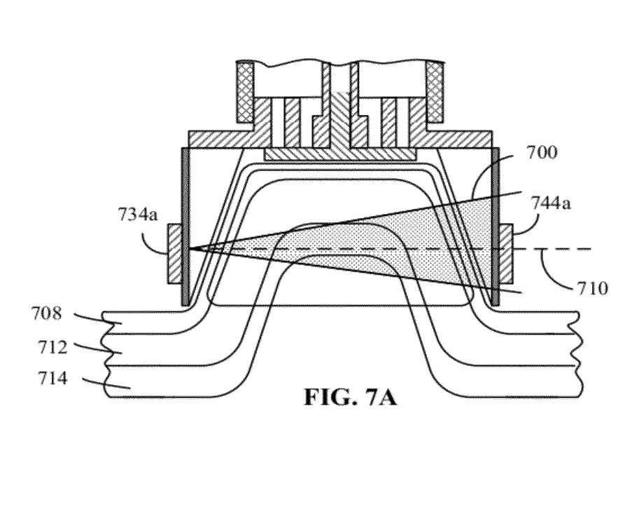

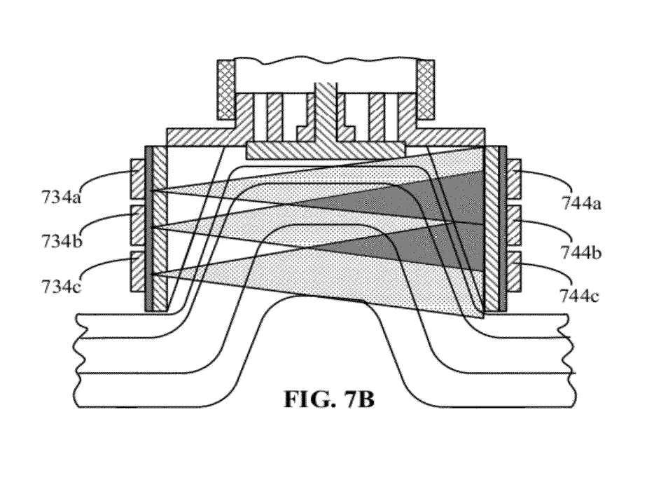

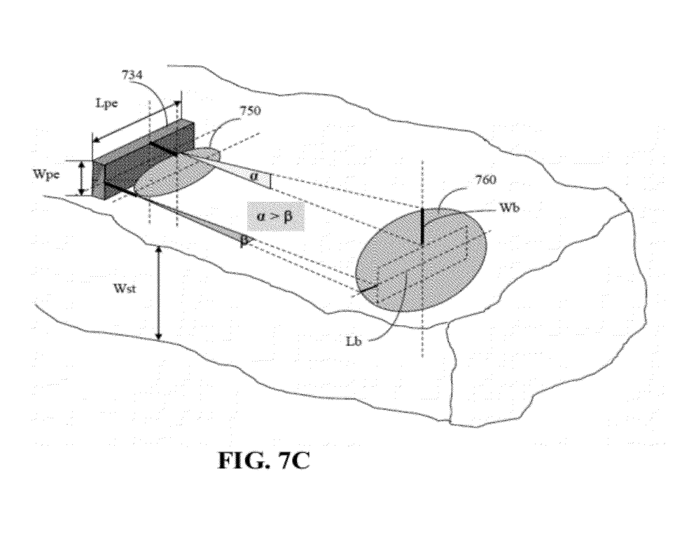

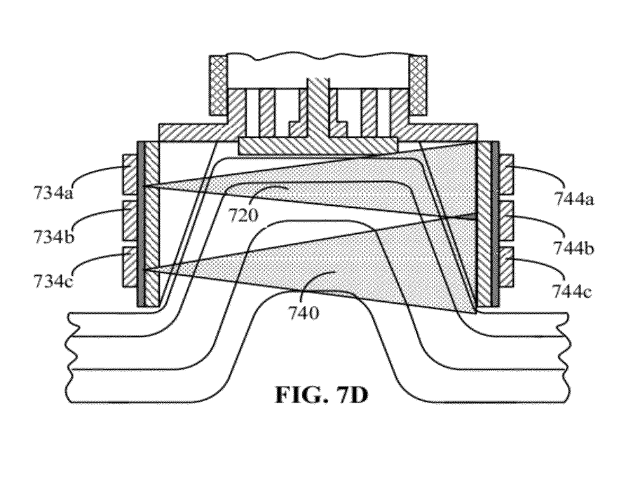

Reference is now made to FIGS. 7A-7D which are simplified views illustrating ultrasound wave propagation in accordance with an exemplary embodiment of the disclosed method and apparatus.

FIG. 7A is a simplified cross-sectional view illustrating an ultrasound beam 700 emitted by a transducer 734a, propagates through a tissue layers 708 and 712 and possibly through other tissue layers and received by transducer 744a. Ultrasound beam 700 does not retain a cylindrical shape, but instead spreads out as it propagates through tissue layer 708 according to basic wave propagation physics laws. Even though beam spread must be taken into consideration, still, the maximum sound pressure is always found along a centerline 710 (acoustic axis) of the transducer.

Beam spread is largely determined by the ultrasound frequency and the surface area dimensions (such as diameter, width and height, etc.) of the emitting surface of the transducer. Beam spread is greater when using a low frequency transducer than when using a high frequency transducer. As the surface area of the transducer emitting surface increases, the beam spread will be reduced.

When employing several piezoelectric elements in a parallel configuration such as elements 334 and 344 illustrated in FIGS. 3A and 3B, beam spread may bring about overlapping of adjacent emitted beams, as illustrated in FIG. 7B and result in interference between emitted ultrasound beams resulting in inaccuracy of the received signals. In accordance with an exemplary embodiment of the disclosed method and apparatus, the ultrasound beams may be emitted in a predetermined sequence at predetermined time intervals, for example, an ultrasound beam is emitted by element 734b first to be received by element 744b, followed by a second beam emitted by element 734a to be received by element 744a, after which a third beam is emitted by element 734c to be received by element 744c. The sequence may be repeated, changed or determined to provide a continuous scanning or sweeping mode, for example, 734a, 734b, 734c, 734a, 734b, 734c and so forth or 734a, 734b, 734c, 734b, 734a, 734b, 734c and so forth. This mode of operation requires a separate driver for each transmitter and/or switching a single driver output between the transducers thus reducing the amount of resources necessary to activate the apparatus. Other embodiments may use beam design which reduces the interference between the transmitted and received beams. Such a design is based on selecting transmitter and receiver dimensions relative to the desired wavelength. The applied voltage by the driver may be in the range between 50V and 1000V, typically between 100V and 500V and more typically between 250V and 350V.

Additionally and alternatively, a beam may be emitted from a single transducer, for example transducer 734b, and received at the same time by transducers (receivers) 744a, 744b and 744c. This allows selection of beam parameters most suitable for the type of tissue being treated and applied treatment protocol.

In accordance with another exemplary embodiment of the disclosed method and apparatus, the piezoelectric elements may be substantially rectangular as illustrated in FIG. 7C, an oblique view illustrating ultrasound wave propagation in accordance with an exemplary embodiment of the disclosed method and apparatus.

The narrow dimension (Wₚₑ) of piezoelectric element 734 is substantially smaller than the length (Lₚₑ) thereof. The acoustic beam emitted by such a rectangular element is shaped by wave diffraction into an elliptical cross section 750 at a distance from element 734 comparable with the size of the element 734. Following this the beam begins to expand along the propagation path. The expansion along the narrow side (Wₚₑ, angle α) is faster than the expansion along the wide side (Lₚₑ, angle β). Divergence angle of the beam depends on the ratio of the plate size to the wavelength. The larger is the ratio the smaller is the divergence angle. When choosing the dimension (Wₚₑ) of the plate, the wavelength have be taken into account, since the speed of sound in the next skin layer outside of Wₛₜ may be higher than in the Wₛₜ layer. Therefore, the signal that propagates into this layer because of beam divergence may reach the receiver earlier than the signal propagating through the layer Wₛₜ. This may lead to measurement errors.

As explained hereinabove, increasing narrow dimension (Wₚₑ) will reduce beam spread thus increasing the resolution of the ultrasound signal received. The value of (Wₚₑ) is determined by the width (Wₛₜ) of the corresponding tissue layer and/or by the distance between elements 734.

It will be appreciated that the external shape of piezoelectric elements 134, 144, 334, 344, 430, 432, 634 and 734 may be of any geometric shape such as oval, triangular, circle, etc. Additionally and alternatively, any two or more piezoelectric elements 134, 144, 334, 344, 430, 432, 634 and 734 in each transducer may differ from each other in size, i.e. length (Lₚₑ), width (Wₚₑ) and thickness in accordance with the transducer elements spatial configuration, type of tissue being treated and selected treatment protocol. In some embodiments the listed piezoelectric elements may be made exchangeable or even disposable.

In accordance with yet another exemplary embodiment of the disclosed method and apparatus, elements 734 may be excited so that no two adjacent elements 734 are exited at the same time. FIG. 7D, which is a simplified cross-sectional view ultrasound wave propagation in accordance with an exemplary embodiment of the disclosed method and illustrates beams 720 and 740 emitted at the same time by corresponding elements 734a and 734c and received by elements 744a and 744c respectively. Elements 734b and 744b are inactivated at this time. This may be followed by element 734b emitting a beam to be received by element 744b. This prevents beam overlapping and interference and increases the accuracy in the information derived from received ultrasound beams. The sequence may be repeated, changed.

Beam spread and the shape of the received beam pulse signal are also affected by the thickness of the piezoelectric element.

It will be appreciated by persons skilled in the art that the present method and apparatus are not limited to what has been particularly shown and described hereinabove. Rather, the scope of the method and apparatus includes both combinations and sub-combinations of various features described hereinabove as well as modifications and variations thereof which would occur to a person skilled in the art upon reading the foregoing description and which are not in the prior art.

What is claimed is:

1. An apparatus for real time monitoring of tissue layers treated by aesthetic body shaping devices, the apparatus comprising:
a housing including:
a first transducer operative to emit ultrasound beams into tissue layers to be treated;
a second transducer, positioned facing said first transducer and sandwiching said tissue layers, operative to receive said ultrasound beams propagated in a substantially direct pathway through said tissue and emitted thereby;
a controller operative to
obtain from said received ultrasound beams information regarding beam signal parameters; and
analyze said information to determine at least one tissue characteristic.

2. The apparatus according to claim 1, and wherein said beam signal parameters are selected from a group consisting of speed of sound, amplitude, frequency and attenuation.

3. The apparatus according to claim 1, and wherein said tissue characteristic is selected from a group consisting of tissue layer identification and change in tissue layer architecture.

4. The apparatus according to claim 1, and wherein the surfaces of said first transducer and second transducer are parallel to each other.

5. The apparatus according to claim 1, and wherein said first transducer and second transducer each also comprise at least one piezoelectric element constructed from at least one piezoelectric material selected from a group consisting of ceramics, polymers and composites.

6. The apparatus according to claim 5, and wherein the thickness of said element (D) is equal or smaller than half the wavelength (λ) at the maximal frequency (f) so that D≦½λ at (fₘₐₓ).

7. The apparatus according to claim 1, and wherein said first transducer and second transducer each also comprise at least two piezoelectric elements positioned in at least one predetermined configuration selected from a group consisting of two-dimensional and three-dimensional spatial configurations.

8. The apparatus according to claim 7, and wherein said elements are constructed from at least one material selected from a group consisting of ceramics, polymers and composites.

9. The apparatus according to claim 7, and wherein a single driver excites said at least two piezoelectric elements.

10. The apparatus according to claim 7, and wherein at least two of said elements in each of said transducers differ from each other in size.

11. The apparatus according to claim 1, and wherein said first transducer and second transducer each also comprise at least one pair of transceivers consisting of
a first transceiver operative to emit ultrasound beams into said tissue layers; and
a second transceiver operative to receive ultrasound beams emitted from said tissue layers.

12. The apparatus according to claim 11, and wherein said first transceiver is also operative to receive ultrasound beams emitted from said tissue layers and said second transceiver is also operative to emit ultrasound beams into said tissue layers.

13. The apparatus according to claim 5, and wherein each of said elements in said first transducer is paired with at least one element in said second transducer.

14. The apparatus according to claim 5, and wherein each of said elements in said first transducer is paired with a corresponding element in said second transducer.

15. The apparatus according to claim 5, and wherein each of said elements in said first transducer is paired with a corresponding element in said second transducer and wherein each pair is positioned to sandwich a substantially discrete tissue layer.

16. The apparatus according to claim 1, and wherein said housing also includes at least one vacuum chamber.

17. The apparatus according to claim 16, and wherein said chamber also comprises walls operative to shift said pathway of said ultrasound beams from a first propagation pathway to a second propagation pathway parallel thereto.

18. The apparatus according to claim 16, and wherein said housing and said chamber also comprise at least one cavity therebetween, and wherein said cavity comprises sound-index matching material operative to minimize acoustic beam attenuation, reflection and refraction.

19. The apparatus according to claim 1, and wherein said tissue layers are a protrusion comprising at least one tissue layer selected from a group consisting of skin, subcutaneous fat and muscle.

20. The apparatus according to claim 16, and wherein said tissue is a protrusion located inside said vacuum chamber and comprising at least one tissue layer selected from a group consisting of skin, subcutaneous fat and muscle.

21. The apparatus according to claim 1, and wherein said first transducer is also operative to emit at least two ultrasound beams along parallel pathways.

22. The apparatus according to claim 1, and wherein said first transducer is also operative to emit at least two ultrasound beams in a predetermined sequence.

23. The apparatus according to claim 21, and wherein said first transducer is also operative to emit said at least two ultrasound beams in a predetermined sequence.

24. The apparatus according to claim 1, and wherein said apparatus also comprises at least one generator operative to excite said first transducer.

25. The apparatus according to claim 1, and wherein said beams are emitted in pulse mode.

26. The apparatus according to claim 1, and wherein said apparatus also comprises at least one amplifier operative to amplify ultrasound beam signals received from said second transducer.

27. The apparatus according to claim 1, and wherein said controller is also operative in real time to:
compare said beam signal parameters and tissue characteristics to a predetermined treatment protocol;
identify changes in said parameters and characteristic and determine the criticality of said changes; and
take at least one action based on said changes and criticality.

28. The apparatus according to claim 27, and wherein said action comprises at least one of the following:
record information relating to said changes and criticality in a database;
display said information on a display;
communicate said changes and criticality to a remote user;
print said information on a printout;
alert a user as to said changes based on said criticality; and
change the course of treatment based on said criticality.

29. The apparatus according to claim 1, and wherein said aesthetic body shaping devices are operative to apply at least one aesthetic body shaping treatment selected from a group consisting of sub-dermal fat cells breakdown, lessening of the amount of sub-dermal fat, tightening of loose skin, tightening and firming of body surfaces, reduction of wrinkles in the skin and collagen remodeling.

30. An apparatus for real time monitoring of tissue layers treated by aesthetic body shaping devices, the apparatus comprising:
a housing including:
a vacuum chamber;
a first transducer operative to emit ultrasound beams into tissue layers inside said chamber;
a second transducer, positioned facing said first transducer and sandwiching said tissue layers, operative to receive said ultrasound beams propagated in a substantially direct pathway through said tissue and emitted thereby;
a controller operative to
obtain from said received ultrasound beams information regarding beam signal parameters; and
analyze said information to determine at least one tissue characteristic.

31. The apparatus according to claim 30, and wherein said apparatus also comprises at least one heating energy delivery surface supplied by a source of heating energy.

32. The apparatus according to claim 31, and wherein said heating energy is in a form of at least one of a group consisting of light, RF, ultrasound, electrolipophoresis, iontophoresis and microwaves.

33. The apparatus according to claim 31, and wherein said first transducer and second transducer also comprise at least one piezoelectric element which is positioned substantially perpendicular to said energy delivery surface.

34. The apparatus according to claim 31, and wherein said first transducer and second transducer each also comprising at least one piezoelectric element and said heating energy delivery surface are positioned on the same plane and adjacent to each other.

35. An apparatus for real time monitoring of tissue layers treated by aesthetic body shaping devices, the apparatus comprising:
a housing including:
a first transducer and a second transducer, each comprising at least two piezoelectric elements said first transducer operative to emit ultrasound beams into tissue layers to be treated and said second transducer, positioned facing said first transducer and operative to receive said beams, and wherein
each element in said second transducer is paired with a corresponding element of said first transducer and positioned to sandwich a substantially discrete tissue layer between them;
a controller operative to
obtain from said received ultrasound beams emitted by said discrete tissue layer information regarding the beam signal parameters; and
analyze said information to determine at least one tissue characteristic.

36. An apparatus for real time monitoring of tissue layers treated by aesthetic body shaping devices, the apparatus comprising:
a housing including:
a vacuum chamber having at least one RF delivery surface operative to deliver RF energy;
a first transducer operative to emit ultrasound beams into tissue layers inside said chamber;
a second transducer, positioned facing said first transducer and sandwiching said tissue layers, operative to receive said ultrasound beams propagated in a substantially direct pathway through said tissue and emitted thereby;
a controller operative to
obtain from said received ultrasound beams information regarding beam signal parameters; and
analyze said information to determine at least one of RF treatment effect and tissue layer type.

37. The apparatus according to claim 36 and wherein said first transducer is also operative to emit ultrasound beams concurrently with the delivery of said RF energy.

38. The apparatus according to claim 36 and wherein said housing also comprises a conductive liquid media conduit operative to externally cool at least one of the surface of said tissue layers and RF delivery surface.

39. A method for real time monitoring of tissue layers treated by aesthetic body shaping devices, the method comprising:
emitting ultrasound beams into tissue layers to be treated;
receiving said ultrasound beams propagated in a substantially direct pathway through said tissue and emitted thereby;
obtaining from said received ultrasound beams information regarding the beam signal parameters; and
analyzing said information to determine at least one tissue characteristic.

40. The method according to claim 39, and wherein said tissue layers are a protrusion comprising at least one tissue layer selected from a group consisting of skin, subcutaneous fat and muscle.

41. The method according to claim 39, and wherein also comprising emitting at least two ultrasound beams along parallel pathways.

42. The method according to claim 39, and wherein also comprising emitting at least two ultrasound beams in a predetermined sequence.

43. The method according to claim 41, and wherein also comprising emitting at least two ultrasound beams in a predetermined sequence.

44. The method according to claim 39, and wherein said ultrasound beams are in pulse form.

45. The method according to claim 39, and wherein also comprising amplifying signals of said ultrasound beams emitted and received.

46. The method according to claim 39, and wherein also comprising receiving ultrasound beams emitted by discrete tissue layers travelled therethrough.

47. The method according to claim 39, and wherein also
comparing said beam signal parameters and tissue characteristic to a predetermined treatment protocol;
identifying changes in said parameters and characteristic and determining the criticality of said changes; and
taking at least one action based on said changes and criticality.

48. The method according to claim 47, and wherein said action comprises at least one of the following:
recording information relating to said changes and criticality in a database;
displaying said information on a display;
communicating said changes and criticality to a remote user;
printing said information on a printout;
alerting a user as to said changes based on said criticality; and
changing the course of treatment based on said criticality.

49. The method according to claim 39, and wherein said treatment applied by said body shaping devices also comprises
breaking down sub-dermal fat cells, lessening the amount of sub-dermal fat, tightening loose skin, tightening and firming body surface, reducing wrinkles in the skin and remodeling collagen.

50. The method according to claim 39, and wherein also comprising applying to said tissue heating energy.

51. The method according to claim 50, and wherein said heating energy is in a form of at least one of a group consisting of light, RF, ultrasound, electrolipophoresis, iontophoresis and microwaves.

52. The method according to claim 50, and wherein also comprising applying said heating energy in a direction substantially perpendicular to the direction of said emitted ultrasound beams.

53. The method according to claim 50, and wherein also comprising applying said heating energy in a direction generally parallel to the direction of said emitted ultrasound beams.

54. A method for real time monitoring of tissue layers treated by aesthetic body shaping devices, the method comprising:
applying RF energy to tissue layers to be treated, then:
emitting ultrasound beams into tissue layers to be treated;
receiving said ultrasound beams propagated in a substantially direct pathway through said tissue and emitted thereby;
obtaining from said received ultrasound beams information regarding the beam signal parameters; and
analyzing said information to determine at least one of RF treatment effect and tissue layer type.

55. The method according to claim 54, and wherein also comprising cooling the tissue layer to be treated.

56. The method according to claim 54, and wherein also comprising concurrently applying, emitting, receiving, obtaining and analyzing.
